(19) United States
(12) Patent Application Publication
(10) Pub. No.: US 2026/0042083 A1
(43) Pub. Date: Feb. 12, 2026

(54) Surface Modified Support for Metal Adsorption and Methods of Using Thereof

(71) Applicant: TDA Research, Inc., Wheat Ridge, CO (US)

(72) Inventors: Gökhan Alptekin, Boulder, CO (US); Freya Kugler, Confier, CO (US); Ewa Jonska-Muteba, Westminster, CO (US); Matthew Schaefer, Lakewood, CO (US); Margarita Dubovik, Demver, CO (US)

(21) Appl. No.: 18/679,181

(22) Filed: May 30, 2024

Related U.S. Application Data
(60) Provisional application No. 63/469,586, filed on May 30, 2023.

(51) Int. Cl. B01J 20/32; B01J 20/22; C02F 1/28; C02F 101/10; C02F 101/20; C07C 7/12

(52) U.S. Cl. CPC B01J 20/3204; B01J 20/22; B01J 20/3219; B01J 20/3251; C02F 1/285; C07C 7/12; C02F 2101/103; C02F 2101/106; C02F 2101/20

(57) ABSTRACT

A sorbent for metal adsorption from liquids, comprising: a porous support material with a surface area of at least 60 m²/g, wherein at least 40% of the pores have a diameter of at least 6 Å, and short chain tether groups with multi-atom chains of at most 8 chain atoms and a metal-binding functional group attached to the support material. Also, a method for removing heavy metal contaminants from liquids, comprising: providing a porous inorganic support material, wherein at least 40% of the pores have a diameter of at least 6 Å, and with a surface area of at least 60 m²/g; providing a plurality of short chain tether groups with multi-atom chains of at most 8 chain atoms and a metal-binding functional group; covalently bonding the tether groups to the support material, forming a sorbent; providing a liquid comprising a heavy metal contaminant; and contacting the liquid to the sorbent.

### RELATED APPLICATIONS

The present application claims the benefit of the provisional application No. 63/469,586 filed May 30, 2023 (titled “Surface Modified Support for metal Adsorption and Methods of Using Thereof”, by Gokhan Alptekin, Margarita Dubovik, Freya Kugler, Ewa Muteba, and Matthew Shaefer, attorney docket number 21-2R), which is incorporated by reference herein.

### STATEMENT OF GOVERNMENT INTEREST

This invention was made using U.S. government funding from the US Navy through the U.S. Naval Air Warfare Center Ad (LKE) Small Business Innovation Research contract, contract number N68335-18-C-0632. The government has certain rights in this invention.

### BACKGROUND

As liquids contact metal-containing materials, such as piping, metal ions (e.g. Cu) can be dissolved in the liquid phase. In the case of liquid hydrocarbon streams, these contaminants can catalyze undesired oxidation reactions that cause decomposition and/or thermal instability of the hydrocarbons. In fuel transport and processing systems, hydrocarbon decomposition may form deposits on the equipment such as fuel lines, valves, injectors, and combustion chamber surfaces. Additionally, the thermal stability of a hydrocarbon fuel may decrease, potentially causing autoignition at lower-than-expected temperatures (a particular problem in jet fuels, such as JP-5).

Dissolved metals, particularly heavy metals, also present a wide range of environmental and human health risks including ground water and drinking water contamination.

Among the purification options, adsorptive removal of dissolved metals (i.e. ions) or any organic metal complexes is used for simple operation, fast adsorption kinetics and ease of integration into existing purification systems. It is complicated to find a successful adsorbent in removing metals from hydrocarbons because it must be highly selective for metal adsorption in the presence of a large excess of various competitive species, such as aromatic and poly-aromatic compounds. The sorbent must also be selective to the metal ions over other chemical additives, including antioxidants, metal deactivators, static reducers, corrosion inhibitors, icing inhibitors, and lubricity improvers, all of which can compete for adsorption sites.

Although there are many high surface area adsorbents, such as alumino-silicates, zeolites, alumina and silica gel, unfortunately, most of the available surface area in these sorbents and hence the adsorption sites reside in their micro-channels. For instance, typical activated alumina has roughly 200-300 m²/g surface area, however, over 60% of this resides in the pores less than 6 Å that are not accessible to the fuel. Because the adsorption is a surface phenomenon, high copper capacity is not achievable with the current adsorbents.

Ion exchange resins with special functional groups have been made for many different applications. For example, DOWEX™ HCR S/S can be used for Ni²⁺ uptake from aqueous solutions. These resins achieve a Ni²⁺ capacity of 20 mg/g (2% wt.) and 40 mg/g (4% wt.) at 10 and 100 ppb concentration in the aqueous phase. Different commercially available resins may have high capacities, up to 235.3 mg/g. (23.5% wt.). However, high capacities in commercial resins are only realized at aqueous concentrations of 100 ppm or higher. Also, the resins typically have a surface area of about 25 m²/g that limits the number of functional groups that can be introduced into the resins and be accessible for Cu and Ni ions. Additionally, many of the resins are designed to work in aqueous systems, and they are far less stable in organics (jet fuel).

### SUMMARY OF THE INVENTION

The present disclosure solves the limitations of the prior art by providing a sorbent with high surface area comprised of mesopores with >6 Å diameter, tethered functional groups that provide an enhanced metal uptake, efficient removal of metals from hydrocarbon liquids via rapid mass transfer into the mesopores. The present disclosure teaches that it is critical to increase the accessible (useful) surface area to achieve a high metal adsorption capacity of metals from liquids with larger molecular sizes, including fuels. The present disclosure provides a sorbent with high capacity for heavy metal contaminants at aqueous concentrations below 100 ppm. The present disclosure teaches both the preferred sorbent for metal adsorption, and the preferred method for adsorbing heavy metal contaminants from aqueous systems such as hydrocarbon fuels and water.

The present disclosure provides sorbent materials, methods of making those sorbent materials, and methods of using those sorbent material for selective removal of metals from liquids. In an embodiment the present invention teaches a sorbent material made from a modified support, with a minimum surface area of 60 m²/g, a minimum pore volume of pores greater than 6 Å of 40%, with metal binding functional groups anchored to a porous support. In certain embodiments the functional group is an —SH group, or an —OH group. The —SH group may be mercaptoacetic acid or other sulfur-containing organic group. In preferred embodiments the functional group is “short” where “short” is defined as having a molecular length of at most 8, 7, 6, 5, or 4 atoms in a continuous chain, when not counting any hydrogen atoms. These molecular lengths also do not count pendant atoms. In certain embodiments the support material may be boehmite, silica, or metal organic frameworks (MOFs). In certain embodiments the sorbent removes Cu, Ni, Pb or other heavy metals from hydrocarbons. In other embodiments the sorbents remove Cu, Ni, Pb, Se, As, or other heavy metals from water. In yet other embodiments the sorbents remove rare earths from aqueous solutions, the rare earth may include Gd, Nd, La, or other rare earths. Other embodiments are methods of using these sorbents to remove heavy metals from aviation fuels, other hydrocarbon fuels, or municipal water.

In other embodiments, the sorbent can remove metals from water. For example: a liquid to sorbent ratio of 1,000:1 and a contact time of 120 minutes (removal of Pb from water from 10 ppm reduced to 7.1 ppm Pb); a liquid to sorbent ratio of 1,000:1 and a contact time of 120 minutes (removal of Se from water from 10 ppm reduced to 1.36 ppm Se); or a liquid to sorbent ratio of 1,000:1 and a contact time of 120 minutes (removal of As from water from 10 ppm reduced to 6.456 ppm As).

The disclosure provides a sorbent for metal adsorption, the sorbent comprising: a support material with a surface area of at least 60 m² per gram of the support material; a plurality of pores within the support material, wherein at least 40% of the pores have a diameter of at least 6 Å; and, a plurality of short chain tether groups attached to the support material, wherein the short chain tether groups comprise multi-atom chains of at most 8 chain atoms and a metal-binding functional group. In some embodiments, the metal-binding functional group comprises a thiol group or a hydroxyl group. In a preferred embodiment, the metal-binding functional group is mercaptoacetic acid. In an embodiment, the support material is mesoporous alumina.

In an optional embodiment, the short chain tether groups comprise at most 6 chain atoms. In another optional embodiment, the short chain tether groups comprise at most 4 chain atoms.

In another embodiment, the support material is a highly active inorganic material. In another embodiment, the support material is selected from the group consisting of: boehmite, silica, or metal organic frameworks.

Preferably, the sorbent does not swell, degrade, break up by attrition, or dissolve during prolonged contact with a fuel.

The disclosure also provides a method for removing heavy metals from liquids, the steps comprising: providing an inorganic support material, wherein the inorganic support material comprises a plurality of pores, wherein at least 40% of the pores have a diameter of at least 6 Å, and wherein the inorganic support material comprises a surface area of at least 60 m² per gram of the support material; providing a plurality of short chain tether groups, wherein the short chain tether groups comprise multi-atom chains of at most 8 chain atoms and a metal-binding functional group; covalently bonding the plurality of short chain tether groups to the inorganic support material, forming a sorbent; providing a liquid comprising a heavy metal contaminant; and, contacting the liquid to the sorbent.

In an embodiment, the liquid has an atomic mass of at least 44 g/mol. The liquid may be a hydrocarbon fuel. In an embodiment where the liquid is a hydrocarbon fuel, the heavy metal contaminant is Cu, Ni, or Pb. In an embodiment where the liquid is water, the heavy metal contaminant is Cu, Ni, Pb, Se, or As.

In a preferred embodiment, there is not a step of removing any organic spacers.

In an embodiment, the liquid is hydrocarbon fuel, the heavy metal contaminant is Cu, and the liquid contacts the sorbent for at most 60 seconds at a ratio of 500:1 (liquid:sorbent).

In an embodiment, the liquid is hydrocarbon fuel, the heavy metal contaminant is Cu, and the liquid contacts the sorbent for at most 60 minutes at a ratio of 10,000:1 (liquid:sorbent).

In an embodiment, the liquid is water, the heavy metal contaminant is Pb, and the liquid contacts the sorbent for at most 120 minutes at a ratio of 1,000:1 (liquid:sorbent).

In an embodiment, the liquid is water, the heavy metal contaminant is Se, and the liquid contacts the sorbent for at most 120 minutes at a ratio of 1,000:1 (liquid:sorbent).

In an embodiment, the liquid is water, the heavy metal contaminant is As, and the liquid contacts the sorbent for at most 120 minutes at a ratio of 1,000:1 (liquid:sorbent).

### BRIEF DESCRIPTION OF FIGURES

FIG. 1. Surface modified mesoporous alumina nanoparticle with polar functional groups (left) and sulfur-carboxylic acid surface modifiers (right).

FIG. 2. Adsorption breakthrough curve showing Cu removal from JP-5 aviation fuel at an inlet concentration of 440 ppb in a flow-through configuration.

FIG. 3. Adsorption curve as a function of contact time showing rare earth removal from water in a batch configuration with a water to sorbent ratio of 50:1.

FIG. 4. Schematic of the formation of mesoporous boehmite nanocomposites.

FIG. 5. XRD pattern and pore size distribution for boehmite nanocomposite.

FIG. 6. The surface modification of mesoporous alumina.

### DETAILED DESCRIPTION OF THE INVENTION

The present disclosure teaches a solid sorbent with a high surface area, a selected pore volume with selected pore sizes, and surface functional groups that enables methods of removing metals (by adsorption) from liquids. In certain embodiments, the disclosure teaches a highly active inorganic support material functionalized with polar surface groups containing sulfur(S). The sorbent is highly active due to the very high surface area of the support material. It is also active due to the high ratio of short-chain functional groups, which avoid steric hindrance between the S molecule and the dissolved metal (even at low concentrations). The sorbent is hydrocarbon-compatible (i.e., it does not dissolve, swell, or lose its mechanical integrity), and retains its functionality in the presence of competing molecules such as aromatic hydrocarbons. The present disclosure also teaches a method of using the sorbent to remove heavy metal contaminant from liquids, such as hydrocarbon fuels or water.

A preferred embodiment of the present invention is a method of removing copper from aviation fuels, non-limiting examples including JP-5 and JP-8. The invention teaches a method that effectively removes the Cu contaminants with very high selectivity and efficiency. A high rate of contaminant removal can be achieved at short liquid-solid contact times, resulting in a compact filter design than can fit into the limited space available in a fuel handling and storage room or on an aircraft carrier. An embodiment of the present invention is a support that is inorganic and that contains surface groups that are covalently bonded to the surfaces (including inside pores) and the material does not swell, degrade, break up by attrition, or dissolve due to prolonged contact with fuel. This solves limitations in the prior art that used a sorbent material to remove copper from JP-5 aviation fuel that were limited by poor mass transfer at the very short liquid-contacts times required for integration into existing fuel systems. Diffusion limitations in the sorbents of the present invention are minimized due to the very high accessible surface area of the mesoporous alumina and high ratio of functional groups attached to the surface, and the small size of those organic groups so that they do not block the pores. In certain preferred embodiments, the liquid to sorbent ratio is 500:1 and the contact time is 60 seconds (for removal of Cu from JP-5 from 475 reduced to less than 10 ppb Cu); alternatively, the liquid to sorbent ratio of 10,000:1 with a contact time of 60 minutes (for removal of Cu from JP-5 from 475 reduced to less than 10 ppb Cu).

Unless defined otherwise, all technical and scientific terms used herein have the same meaning as commonly understood by one of ordinary skill in the art to which the invention belongs. All abbreviations used in the description of the invention are described in the embodiments.

The term “comprises” and grammatical equivalents thereof are used herein to mean that other components, ingredients, steps, etc. are optionally present. For example, an article “comprising” (or “which comprises”) component A, B, and C can consist of (i.e. contain only) components A, B, and C, or can contain not only components A, B, and C but also one or more other components.

The term “at least” followed by a number is used herein to denote the start of a range beginning with that number (which may be a range having an upper limit or no upper limit, depending on the variable being defined). For example, “at least 1” means 1 or more than 1. The term “at most” followed by a number is used herein to denote the end of a range ending with that number (which may be a range having 1 or 0 as its lower limit, or a range having no lower limit, depending on the variable being defined). For example, “at most 8” means 8 or less than 8, and “at most 40%” means 40% or less than 40%. When, in this specification, a range is given as “(a first number) to (a second number)” or “(a first number)-(a second number)”, this means a range whose lower limit is the first number and whose upper limit is the second number. For example, “2 to 10 atoms” means a range whose lower limit is 2 atoms, and whose upper limit is 10 atoms.

The term “short chain tether group” is defined as a linear multi-atom chain that comprises 8 or fewer chain atoms. It may comprise 8, 7, 6, 5, 4, 3, or 2 chain atoms. The chain atoms are the linearly connected core atoms which form the longest continuous series of atoms, not counting hydrogens, pendant atoms, or shorter branches off the longest linear chain. The chain atoms may be carbons. In a non-limiting example, the short chain tether group is a carboxylic acid chain with a thiol or hydroxyl group at one end for binding with metal contaminants. The short chain tether group must covalently bond with the surface support material, for example carboxylic groups attaching to —OH groups on the surface.

The term “metal-binding functional group” is defined as a polar molecular group capable of interacting with metal impurities contained in a liquid, such as a liquid hydrocarbon fuel. The functional group may be selected for a specific metal impurity, such as copper—in the case of copper, thiol groups or thiolic acids are preferred metal-binding functional groups due to their copper ion activity. The metal-binding functional group does not need to covalently bond the metal ions in solution, but the strength of the interaction must overcome Van der Waals forces and any other forces keeping the metal ions in solution.

The term “heavy metal contaminant” is defined as a dissolved metal that potentially threatens human or machine health or safety (e.g., unsafe groundwater or drinking water, decreased thermal stability of fuels). Examples of heavy metal contaminants include Cu, Ni, Pb, Se, As, and the like.

Cab-O-Sil® is an aerosol silica that can be mixed with most liquids to form a thick colloidal suspension, and is a light, fluffy white powder made by hydrogen-oxygen furnace combustion of silicon tetrachloride. Catapal® is a group of high-purity alumina hydrates that are traditionally used as supports or binders. Versal® is a family of high-performance alumina powders and includes products such as pseudoboehmite, bayerite, gamma and alpha aluminas, and have varying density, porosity, acid stability, and thermal sintering characteristics.

The present disclosure teaches a method for using surface modified sorbents to remove metals, such as copper, from aviation fuel. As jet fuel flows through the CuNi fuel lines in aircraft fueling system and subsystems, copper is dissolved into the fuel in small but finite amounts. Copper contamination in the fuel can reach levels as high as 1 ppmw. The copper contaminants in fuel catalyzes oxidation reactions that cause hydrocarbons to decompose to elemental carbon and form deposits on the fuel lines, valves, injectors, and combustion chamber surfaces in engines. It is the organic acid and polar compounds in the fuel that are responsible in dissolving the copper from the pipes, altering the fuel properties by eliminating these species to reduce copper leaching is not practical because these polar compounds constitute up to 30% of the fuel volume. Hence, there is a need for technologies to filter out the dissolved copper particles from the fuel.

The present disclosure teaches a new material that consists of a high surface area support material functionalized with thiol-containing polar groups. In the functionalization, the anchor groups are carboxylic acids that will react with the surface hydroxyl groups (—OH), while their —SH functional groups stick out to interact with the metal impurities.

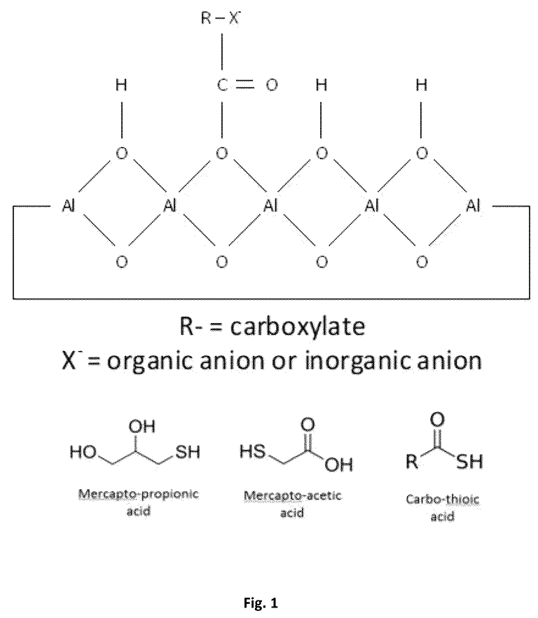

The present disclosure teaches mesoporous functionalized alumina structures (sorbents) that can be decorated with functional groups. Our materials have high surface area, properly sized (selected as taught in this invention) pores, and are stable in organics. FIG. 1 shows a schematic of a boehmite nanoparticle containing carboxylate groups that complexes with an inorganic cation. These alumina supports, when modified with the correct surface functionality, could allow the sorbent to achieve a high copper removal efficiency (i.e., reducing the copper content in the fuel to <10 ppb levels). To support the chelating interactions, the surface of a high surface area support can be modified by functional groups that are active to copper ion adsorption (e.g., by bonding the surface to carbon chains containing functional groups like aminos, carboxyls, sulfonics, cyclams, DETAs, anhydrides and lactones).

The present disclosure teaches various surface functionalities tethered onto very high surface area support material (with mesopores), which are attached by chemically attaching short-chain polar groups containing —SH, such as mercaptopropionic acid (MPA), mercaptoacetic acid (MAA), and carbo-thioic acid to the —OH groups on the support surface. These carbo-thiolic groups are small with minimal steric restrictions (to not further reduce the internal pore sizes) and highly accessible, allowing them to interact with the metal ions in liquid-phase via a chemical complexation process. Although not wishing to be bound by theory, in certain embodiments the S atoms in these groups have a pair of unshared electrons that can complex with the metals by donating electrons to the electron-deficient metal ion. And further while not wishing to be bound by theory, although the surface groups and the metal ions do not form a true covalent bond, the interaction is much stronger than that observed in physical adsorption systems dominated by Van der Waal type interactions. The heat of adsorption of Cu over the S functional groups, for example, is greater than 10 kcal/mol, 7-8 times higher than typical Van der Waal type interactions. This strong affinity towards the metal ions generates the needed selectivity that allows removal of metal impurities in low concentrations, even in the presence of competitive species including unsaturated hydrocarbons (such as olefins, aromatics and polyaromatics).

The polar-functionalized material of the present disclosure can be produced by a process that includes anchoring the first reactant (thiol) with the second reactant (hydroxide-containing inorganic support). The combined reactants are mixed and potentially heated to cause a reaction between the first and second reactants, eventually forming the product. This process can include the addition of one or more solvents, formation of a hydrogel, a second reaction step, rinsing with one or more solvents, and a subsequent curing or dehydration step.

Example 1: In a 1 L round bottom flask equipped with an overhead mechanical stirrer and a reflux condenser, 25 g (0.19 mol Al) of boehmite with a BET surface area of over 200 m²/g (e.g. Catapal A from Sasol) was slurried in 300 g of water. To this was slowly added 6 g (0.07 mol) of thioglycolic acid while stirring vigorously, adding additional water as necessary to maintain a low enough viscosity to ensure good mixing. This mixture was heated to 80° C. for 24 hours at ambient pressure and allowed to dry at ambient conditions in a thin (<⅛″) layer until the loss on drying exceeded 90%.

In this example, the thioglycolic acid (also known as mercaptoacetic acid) contains a carboxylic acid end as the anchor group and a thiol molecule as the polar functional group. The support material is mesoporous alumina.

Example 2: In a 1 L round bottom flask equipped with an overhead mechanical stirrer and a reflux condenser, 25 g (0.19 mol Si) of fumed silica with a BET surface area of over 200 m²/g (e.g. Cab-O-Sil® from Cabot Corp.) was slurried in 800 g of water. To this was slowly added 6 g (0.07 mol) of thioglycolic acid while stirring vigorously, adding additional water as necessary to maintain a low enough viscosity to ensure good mixing. This mixture was heated to 95° C. for 15 hours at ambient pressure and allowed to dry at room temperature for 1 week.

In this example, the thioglycolic acid contains a carboxylic acid end as the Anchor group and a thiol as the polar functional group. The support material is fumed silica.

Other formulations prepared by these methods may substitute the following thiolic-acids as the functional group: mercaptopropionic (MPA), carbo-thioic acid, mercaptopropyltrimethoxysilane (MPTMS), or other similar short-chain acids. Other formulations prepared by this method may substitute any support material containing hydroxide surface groups (—OH), such as: Catapal® B from Sasol, Versal® V700, Versal® V900, SA6176 aluminum oxide, 13X alumina silicate, Cab-O-Sil® from Cabot (or equivalent), silicic acid, titania, zirconia, UiO66, MOF-808. A summary of the contaminant-removal performance of the material is shown in Table 1.

TABLE 1
Experimental results for cation impurity removal from liquids.

| | | | | Liquid to | | | |
|---|---|---|---|---|---|---|---|
| | | | Sample | Sorbent | Contact | Final impurity | Removal |
| Impurity | Matrix | Combination | Number | Ratio | time | concentration | Efficiency |
| 475 ppb Cu | JP-5 | MAA/ | 1292-40A | 500:1 | 60 | <10 ppb Cu | 98% |
| | | Catapal ® A | | | seconds | | |
| 475 ppb Cu | JP-5 | MAA/Cab- | 1260-10B | 10,000:1 | 60 | <10ppb Cu | 98% |
| | | O-Sil ® | | | minutes | | |
| 10 ppm Pb | Water | MAA/ | 1230-94 | 1,000:1 | 120 | 7.10 ppm | 29% |
| | | Catapal ® A | | | minutes | | |
| 10 ppm Se | Water | MAA/UiO66 | 1306-49 | 1,000:1 | 120 | 1.36 ppm | 86% |
| (as selenate) | | | | | minutes | | |
| 10 ppm As | Water | MAA/UiO66 | 1306-49 | 1,000:1 | 120 | 6.45 ppm | 36% |
| | | | | | minutes | | |
| 672 ppm Gd, | Water | MAA/ | 1314-55 | 50:1 | 20 | 0.5 ppm Gd, | 100% |
| 617 ppm Nd, | | Catapal ® A | | | hours | 0.5 ppm Nd, | |
| 1034 ppm La | | | | | | 0.6 ppm La | |

FIG. 1 shows a generic scheme for the formation of an alumoxane hydrogel functionalized with a polar surface group as well as potential sulfur-carboxylic acid surface modifiers.

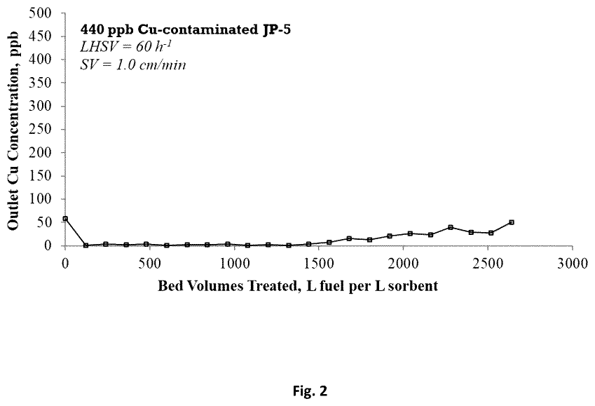

Example 3: In a fuel purification application, the material can be used to remove low concentrations of Cu from aviation fuel (JP-5) or other liquid hydrocarbons. At a contact time of 60 seconds in an up-flow configuration, the sorbent reduces the inlet Cu concentration of JP-5 from 440 ppb to less than 10 ppb in the treated fuel. Over 1,500 bed volumes of fuel can be treated in this manner before any Cu slippage occurs, and contaminant breakthrough does not occur until at least 2,500 bed volumes have been passed through the packed sorbent bed as shown in FIG. 2. The saturation capacity of the sorbent has been measured in excess of 0.35% wt. Cu in batch experiments at a fuel to sorbent ratio of 10,000:1 where the starting fuel contains 475 ppb Cu and the outlet concentration is reduced to <40 ppb Cu.

Example 4: In water applications, the material can be used to remove toxic heavy metals such as Cu, Pb, Se, and As that are common contaminants in coal combustion residual leachates and drinking water piping infrastructure. The removal efficiency of the sorbent material was evaluated for these contaminates in a 500 ml flask containing 250 ml of water containing 10 ppm of a single metal combined with 250 mg of sorbent and stirred at room temperature with a magnetic stir bar for 2 hours. After 2 hours, the treated water was filtered using a #5 Watman paper filter using a Buchner funnel and vacuum flask. The filtrate was then analyzed for the remaining metal using an Agilent 4200 MP-AES. The results are reported in Table 1.

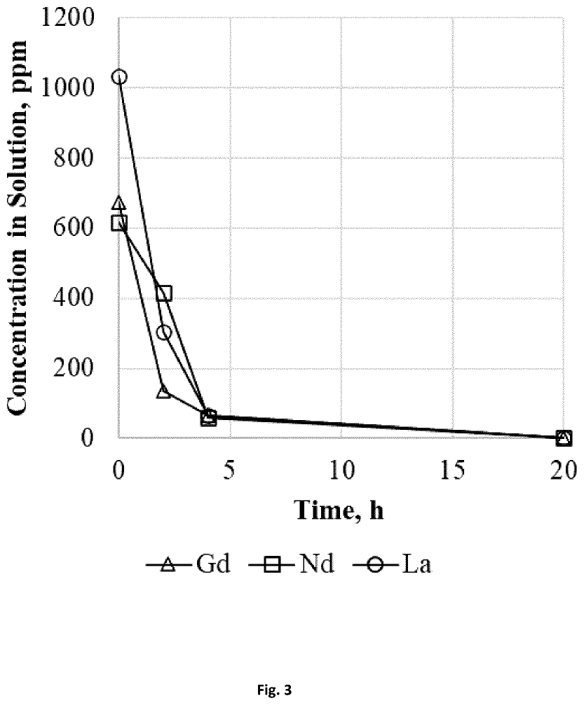

Example 5: In rare earth element (REE) recovery applications, the material can be used as an absorbent to remove REE's from aqueous solutions with a capacity of at least 0.12% wt. An aqueous solution containing 100 g of deionized water, 671 ppm Gd, 617 ppm Nd, and 1033 ppm La was combined in a 200 ml flask with 2 g of sorbent and stirred for 20 hours at room temperature. A 1 ml aliquot was decanted from the top of the flask using a pipette after 1 hour, 4 hours, and 20 hours and filtered using a 0.45 micron PTFE syringe filter. Each sample was analyzed for Gd, Nd, and La using an Agilent 4200 MP-AES. After 20 hours, the concentration of all three REE's was found to be <1 ppm each, for an overall removal efficiency of 99.9%. A summary of the results is shown in FIG. 3 and Table 1.

Example 6: In a comparative example, substituting a longer chain tether group, diethylenetriamine (DETA, C₄H₁₃N₃), for thioglycolic acid in the preparation described in Example 1 produced a material that was not as effective at removing Cu from JP-5 aviation fuel. DETA is well-documented as an effective chelating agent for removing Cu from aviation fuel, and polyamine chelants are among the strongest metal chelators known (Morris, 2015). When exposed to JP-5 containing 475 ppb Cu for 60 minutes at a liquid to sorbent ratio of 10,000:1, the removal efficiency of the DETA-functionalized material was only 58% with a final impurity concentration of 197 ppb Cu; as compared to 90% removal efficiency with a final impurity concentration of <10 ppb Cu under the same conditions.

The carboxyl, anhydride, and lactone groups supported on activated carbons are active for the adsorption of Cu²⁺ ions. Other chelators, such as 1,4,8,11-tetraaza-cyclotetra decane (cyclam) and N¹-[3-(trimethoxysilyl)propyl]diethylenetriamine (DETA), also works well for Cu²⁺ removal. However, the capacity of the carbon filters is limited, resulting in very large size filters. The use of soluble chelating agents causes other problems, forming gelatinous precipitates that can clog lines and valves. In certain embodiments the present invention teaches methods that avoid these limitations.

In contrast, functional groups are dispersed on a high surface area support such as alumina or silica the sorbent's sorption capacity can be significantly increased.

There are a large number of modifiers that can be used to further improve the sorbent's performance. Sulfided carboxylic acids groups anchored to boehmite surface are a preferred material for Cu removal. Smaller groups (with 8, 7, 6, 5, or preferably 4 atoms in the longest continuously bonded chain of atom in the molecule, not counting hydrogen atoms) provide higher removal rates due to reduced transport limitations inside pores.

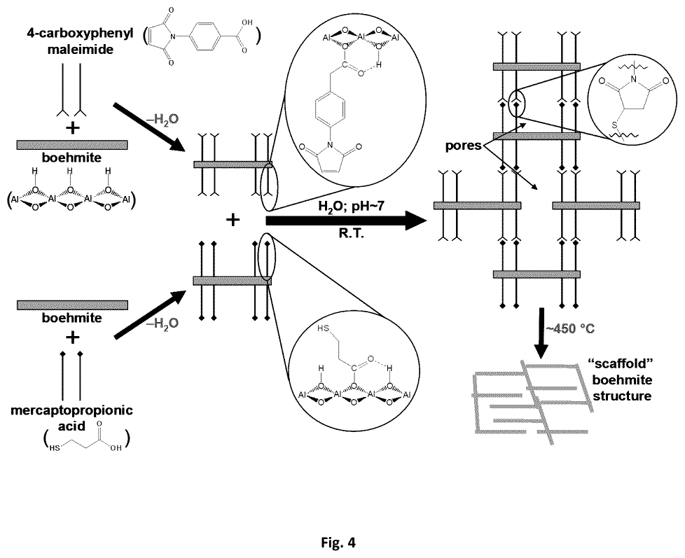

If one type of functional group is first made and then a second modified boehmite nanoparticles is made with a second type of functional groups, it is possible to cause the two types of surface-modified nanoparticles to self-assemble into a novel mesoporous structure. A preferred reaction is between thiols and maleimides. The reaction between thiols and maleimides occurs rapidly in water at room temperature and at neutral pH. Mixing neutral room temperature solutions of mercaptopropionic acid surface modified boehmite nanoparticles with 4-CPMI-surface modified boehmite nanoparticles results in the self-assembly of mesoporous organoboehmite nanostructures (FIG. 4). Essentially, we cover the surface of some boehmite nanoparticles with 4-CPMI and the surface of others with 3-mercaptopropionic acid. When we mix them together they snap (react) together to form scaffold-like lattice. By using excess thiol the resulting material has tethered thiol groups inside the mesopores defined by the bridging groups that are bonded together during self-assembly.

In contrast to all other mesoporous alumina (or silica/silica-alumina) materials the material of the present invention can be made without the need for thermal treatment to remove the organic spacers.

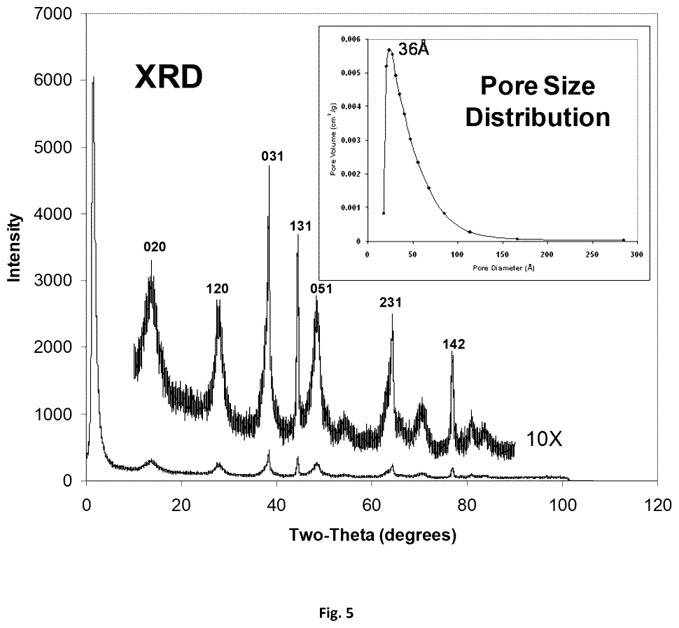

FIG. 5 shows an X-ray diffraction (XRD) pattern of the material. The strong peak at small two-theta (1.55°) is indicative of long-range order typical of mesoporous materials and BJH analysis shows an average pore diameter of 36 Å.

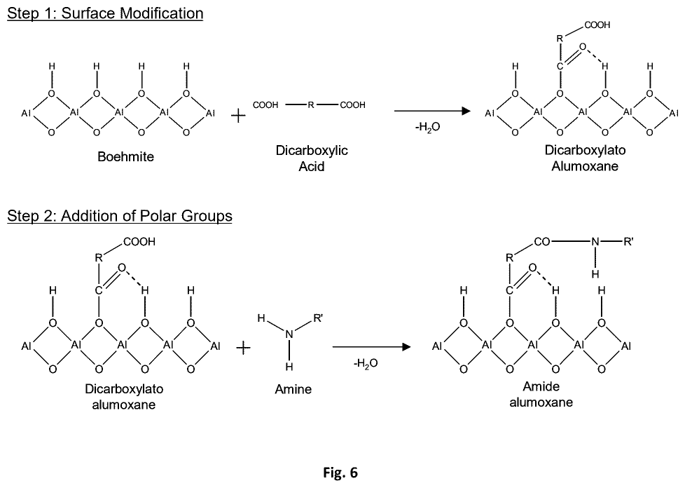

In one embodiment the mesoporous alumina with the organic groups used as pillars between alumina layers are electron rich and interact with the electron deficient copper ions to form a complex. In other embodiments alumina is modified using carboxylic acids having other functional groups (FIG. 6). For example, the surface can be made hydrophobic using trifluoro-acetic acid. Amino-acetic acid can be added to the surface to make the amine-functional. Cyanoacetic acid can be attached to the surface to provide a highly polar group. A list of potential functional groups that can be added to the mesoporous alumina support is provided in Table 2.

The mesoporous boehmite (alumina) can then be modified by adding polar nitrogen groups like-CN, —CH—NH or —CH₂—NH₂. These groups interact with the copper ion via a complexation process. The N atom in these ligands has a pair of unshared electrons that can complex with the copper by donating electrons to the electron-deficient metal ion. While the surface groups and the copper ions do not form a true covalent bond, the interaction is much stronger than that observed in physical adsorption systems dominated by Van der Waals type interactions. The heat of adsorption of copper over the proposed amine functional groups is greater than 10 kcal/mol, 7-8 times higher than typical Van der Waal type interactions. This strong affinity towards the copper ions generates the needed selectivity that allows removal of copper ions in very small concentrations, even in the presence of unsaturated hydrocarbons (such as olefins, aromatics and polyaromatics).

TABLE 2
Polar alumina structures.

| | Surface groups |
|---|---|
| Alumina | —AlOH |
| Diol | —Al—(CH₂)₃—O—CH₂—CH₂(OH) |
| Cyanopropyl | —Al—(CH₂)₃—CN |
| Amide | —Al—(CH₂)ₙ—CO—NH₂ |
| Amine | —Al—(CH₂)ₙ—NH₂ |

In an exemplary embodiment, the disclosure provides a sorbent with high surface area (at least 60 m²/g) in the >6 Å size, metal adsorption, wherein the surface area is defined by pores that are large enough to be accessible liquids with larger atomic mass (44 g/mol or greater), non-limiting examples including aviation or vehicle fuels.

The disclosure provides sorbent materials having surface tethered functionalities on high surface area support materials (at least 50 m² per gram, at least 55 m² per gram, at least 57 m² per gram, at least 60 m² per gram, at least 62 m² per gram, at least 65 m² per gram, at least 67 m² per gram, at least 70 m² per gram), at in the form of short-chain polar groups containing a —SH, such as mercaptopropionic acid (MPA), mercaptoacetic acid (MAA), and carbo-thioic acid, which is tethered, via bonding, to the —OH groups on the support surface. These carbo-thiolic groups are “small” with minimal steric restrictions (to not further reduce the internal pore sizes) and highly accessible, allowing them to interact with the metal ions in liquid-phase via a chemical complexation process. The functional group is “small” or “short” where “small” (alternatively called “short”) is defined as having a molecular length of at most 8, 7, 6, 5, or 4 atoms long, when not counting any hydrogen atoms. These molecular lengths also do not count pendant atoms not in the longest chain. The S atom in these groups has a pair of unshared electrons that can complex with the metals by donating electrons to the electron-deficient metal ion.

The disclosure provides a sorbent material made from a modified support, with a minimum surface area of at least 60 m²/g, a minimum pore volume of pores greater than 6 Å of 40%, with metal binding functional groups anchored to a porous support. In certain embodiments the functional group is an —SH group, or an —OH group. The —SH group may be mercaptoacetic acid or other sulfur-containing organic group. In preferred embodiments the functional group is “short” where “short” is defined as having a molecular length of at most 8, 7, 6, 5, or 4 atoms long, when not counting any hydrogen atoms. These molecular lengths also do not count pendant atoms. The support material may be boehmite, silica, or metal organic frameworks (MOFs). The sorbent may remove Cu, Ni, Pb or other heavy metals from hydrocarbons. Alternatively, the sorbents may remove Cu, Ni, Pb, Se, As, or other heavy metals from water.

The disclosure provides a method of removing copper from aviation fuels, with a minimum rate of contaminant removal at a maximum liquid-solid contact times, resulting in a compact filter design that can fit into the limited space available in a fuel handling and storage room or on an aircraft carrier. The support is inorganic and the surface groups are covalently bonded to the surface, so the material does not swell, degrade, break up by attrition, or dissolve due to prolonged contact with fuel. Diffusion limitations in the sorbents are minimized due to the very high accessible surface area of the mesoporous alumina and high ratio of functional groups attached to the surface. Particular embodiments are:

A liquid to sorbent ratio of 500:1 and a contact time of 60 seconds (removal of Cu from JP-5 from 475 reduced to less than 10 ppb Cu).

A liquid to sorbent ratio of 10,000:1 and a contact time of 60 minutes (removal of Cu from JP-5 from 475 reduced to less than 10 ppb Cu).

A liquid to sorbent ratio of 1,000:1 and a contact time of 120 minutes (removal of Pb from water from 10 ppm reduced to 7.1 ppm Pb).

A liquid to sorbent ratio of 1,000:1 and a contact time of 120 minutes (removal of Se from water from 10 ppm reduced to 1.36 ppm Se).

A liquid to sorbent ratio of 1,000:1 and a contact time of 120 minutes (removal of As from water from 10 ppm reduced to 6.456 ppm As).

What is claimed is:

1. A sorbent for metal adsorption, the sorbent comprising:
a) a support material with a surface area of at least 60 m² per gram of the support material;
b) a plurality of pores within the support material, wherein at least 40% of the pores have a diameter of at least 6 Å; and,
c) a plurality of short chain tether groups attached to the support material, wherein the short chain tether groups comprise multi-atom chains of at most 8 chain atoms and a metal-binding functional group.

2. The sorbent as in claim 1, wherein the metal-binding functional group comprises a thiol group or a hydroxyl group.

3. The sorbent as in claim 2, wherein the metal-binding functional group is mercaptoacetic acid.

4. The sorbent as in claim 3, wherein the support material is mesoporous alumina.

5. The sorbent as in claim 1, wherein the short chain tether groups comprise at most 6 chain atoms.

6. The sorbent as in claim 5, wherein the short chain tether groups comprise at most 4 chain atoms.

7. The sorbent as in claim 1, wherein the support material is a highly active inorganic material.

8. The sorbent as in claim 1, wherein the support material is selected from the group consisting of:
boehmite, silica, or metal organic frameworks.

9. The sorbent as in claim 1, wherein the sorbent does not swell, degrade, break up by attrition, or dissolve during prolonged contact with a fuel.

10. A method for removing heavy metals from liquids, the steps comprising:
a) providing an inorganic support material, wherein the inorganic support material comprises a plurality of pores, wherein at least 40% of the pores have a diameter of at least 6 Å, and wherein the inorganic support material comprises a surface area of at least 60 m² per gram of the support material;
b) providing a plurality of short chain tether groups, wherein the short chain tether groups comprise multi-atom chains of at most 8 chain atoms and a metal-binding functional group;
c) covalently bonding the plurality of short chain tether groups to the inorganic support material, forming a sorbent;
d) providing a liquid comprising a heavy metal contaminant; and,
e) contacting the liquid to the sorbent.

11. The method as in claim 10, wherein the liquid has an atomic mass of at least 44 g/mol.

12. The method as in claim 11, wherein the liquid is a hydrocarbon fuel.

13. The method as in claim 12, wherein the liquid is hydrocarbon fuel and the heavy metal contaminant is Cu, Ni, or Pb.

14. The method as in claim 10, wherein the liquid is water and the heavy metal contaminant is Cu, Ni, Pb, Se, or As.

15. The method as in claim 10, wherein there is not a step of removing any organic spacers.

16. The method as in claim 10, wherein the liquid is hydrocarbon fuel, the heavy metal contaminant is Cu, and step e) comprises contacting the liquid to the sorbent for at most 60 seconds at a ratio of 500:1 (liquid:sorbent).

17. The method as in claim 10, wherein the liquid is hydrocarbon fuel, the heavy metal contaminant is Cu, and step e) comprises contacting the liquid to the sorbent for at most 60 minutes at a ratio of 10,000:1 (liquid:sorbent).

18. The method as in claim 10, wherein the liquid is water, the heavy metal contaminant is Pb, and step e) comprises contacting the liquid to the sorbent for at most 120 minutes at a ratio of 1,000:1 (liquid:sorbent).

19. The method as in claim 10, wherein the liquid is water, the heavy metal contaminant is Se, and step e) comprises contacting the liquid to the sorbent for at most 120 minutes at a ratio of 1,000:1 (liquid:sorbent).

20. The method as in claim 10, wherein the liquid is water, the heavy metal contaminant is As, and step e) comprises contacting the liquid to the sorbent for at most 120 minutes at a ratio of 1,000:1 (liquid:sorbent).
